(19) United States
(12) Patent Application Publication
(10) Pub. No.: US 2023/0397886 A1
(43) Pub. Date: Dec. 14, 2023

(54) INFORMATION PROCESSING APPARATUS, INFORMATION PROCESSING SYSTEM, INFORMATION PROCESSING METHOD, AND INFORMATION PROCESSING PROGRAM

(71) Applicant: FUJIFILM CORPORATION, Tokyo (JP)

(72) Inventors: Yasuhisa KANEKO, Kanagawa (JP); Tomohide HIRAGAMI, Kanagawa (JP); Kenji NAGAMIYA, Kanagawa (JP); Nobuya KITAMURA, Kanagawa (JP); Yasuyuki HOSONO, Kanagawa (JP)

(21) Appl. No.: 18/455,572

(22) Filed: Aug. 24, 2023

Related U.S. Application Data
(63) Continuation of application No. PCT/JP2022/009256, filed on Mar. 3, 2022.

(30) Foreign Application Priority Data
Mar. 5, 2021 (JP) 2021-035801

(51) Int. Cl. A61B 5/00

(52) U.S. Cl. CPC A61B 5/7228; A61B 5/7264

(57) ABSTRACT

An information processing apparatus comprising at least one processor, wherein the processor is configured to: acquire first information including biological information of a user; determine whether or not the biological information included in the first information satisfies a predetermined condition; and issue, to a measurement apparatus that measures the biological information of the user and that is not associated with the information processing apparatus, a command regarding the measurement according to a result of the determination.

### CROSS REFERENCE TO RELATED APPLICATIONS

This application is a continuation of International Application No. PCT/JP2022/009256, filed on Mar. 3, 2022, which claims priority from Japanese Patent Application No. 2021-035801, filed on Mar. 5, 2021. The entire disclosure of each of the above applications is incorporated herein by reference.

### BACKGROUND

### Technical Field

The present invention relates to an information processing apparatus, an information processing system, an information processing method, and an information processing program.

### Related Art

In the related art, a technique for efficiently performing diagnosis, treatment, health management, and the like by controlling various devices according to certain biological information is known. For example, JP2016-502420A discloses that a hypoglycemia indicator used for treatment is activated in a case where a real-time glucose value satisfies a threshold value.

In recent years, biological information of a user has been acquired by thermometers, sphygmomanometers, urine examination apparatuses, and the like disposed in public places. With the proliferation of wearable terminals such as smart watches, there is an increasing opportunity for a user to daily acquire biological information such as a heart rate and arterial blood oxygen saturation. By acquiring biological information on a daily basis in this way, it is possible to detect a sign of deterioration of a medical condition at an early stage and to utilize the biological information for health management.

Meanwhile, in a case where the user is healthy, it is troublesome for the user to continue to acquire the biological information, or the power consumption of a biological information measurement apparatus increases, which is not preferable in some cases. Therefore, for example, a technique that enables acquisition of various kinds of biological information as necessary according to a health condition of a user is desired.

### SUMMARY

The present disclosure provides an information processing apparatus, an information processing system, an information processing method, and an information processing program capable of acquiring biological information as necessary according to a health condition of a user.

According to a first aspect of the present disclosure, there is provided an information processing apparatus comprising at least one processor, in which the processor acquires first information including biological information of a user, determines whether or not the biological information included in the first information satisfies a predetermined condition, and issues, to a measurement apparatus that measures biological information of the user and that is not associated with the information processing apparatus, a command regarding the measurement according to a result of the determination.

According to a second aspect of the present disclosure, in the above aspect, the first information may include a plurality of pieces of different types of biological information, the condition may be predetermined for each type of the biological information, and the processor may determine whether or not at least one of the plurality of pieces of biological information included in the first information satisfies the condition.

According to a third aspect of the present disclosure, in the above aspects, the processor may issue, to each of a plurality of measurement apparatuses that measure the same type of biological information, the command according to the result of the determination.

According to a fourth aspect of the present disclosure, in the first aspect or the second aspect, the processor may issue, to each of a plurality of measurement apparatuses that measure different types of biological information, the command according to the result of the determination.

According to a fifth aspect of the present disclosure, in the above aspects, the command may be an instruction to cause the measurement apparatus to measure the biological information of the user.

According to a sixth aspect of the present disclosure, in the above aspects, the command may be an instruction to transmit the biological information of the user measured by the measurement apparatus to the information processing apparatus.

According to a seventh aspect of the present disclosure, in the sixth aspect, the processor may acquire the biological information of the user measured by the measurement apparatus in response to the command, determine whether or not the biological information satisfies the predetermined condition, and issue, to a measurement apparatus different from the measurement apparatus, a command regarding the measurement of the biological information of the user according to a result of the determination.

According to an eighth aspect of the present disclosure, in the above aspects, the processor may receive designation by the user for at least one of a type of the biological information used for the determination or a type of biological information of a candidate for measurement by the measurement apparatus.

According to a ninth aspect of the present disclosure, in the above aspects, the processor may receive a user's intention regarding health management of the user, and set at least one of a type of the biological information used for the determination or a type of biological information of a candidate for measurement by the measurement apparatus according to the intention.

According to a tenth aspect of the present disclosure, in the above aspects, the condition may relate to a predetermined threshold value for the biological information included in the first information.

According to an eleventh aspect of the present disclosure, in the tenth aspect, a plurality of threshold values may be defined in a stepwise manner, and the processor may determine a classification of the biological information included in the first information using the plurality of threshold values and may issue the command according to the classification.

According to a twelfth aspect of the present disclosure, in the above aspects, the processor may establish connection with the measurement apparatus before issuing the command to the measurement apparatus and may release the connection with the measurement apparatus after issuing the command to the measurement apparatus.

According to a thirteenth aspect of the present disclosure, in the above aspects, the processor may search for the measurement apparatus present near a current location of the user and may issue the command to the measurement apparatus.

According to a fourteenth aspect of the present disclosure, in the above aspects, the processor may settle a usage fee before issuing the command to the measurement apparatus.

According to a fifteenth aspect of the present disclosure, in the above aspects, the biological information may indicate at least one of a body temperature, a heart rate, an electrocardiogram, an electromyogram, blood pressure, arterial blood oxygen saturation, a weight, a body fat percentage, a muscle mass, a bone density, a blood glucose level, a result of a hematological examination, a result of an infectious disease examination, a result of a biochemical examination, or a result of a urine examination.

According to a sixteenth aspect of the present disclosure, the information processing apparatus according to any one of the above aspects may further comprise a first measurement apparatus that measures biological information of the user, in which the processor may acquire the first information including the biological information of the user measured by the first measurement apparatus.

According to a seventeenth aspect of the present disclosure, there is provided an information processing system comprising: the information processing apparatus according to the sixteenth aspect; and at least one second measurement apparatus that measures biological information of the user in response to a command from the information processing apparatus.

According to an eighteenth aspect of the present disclosure, there is provided an information processing system comprising: the information processing apparatus according to any one of the first to fifteenth aspects; at least one first measurement apparatus that measures biological information of the user and that transmits the first information including the measured biological information to the information processing apparatus; and at least one second measurement apparatus that measures biological information of the user in response to a command from the information processing apparatus.

According to a nineteenth aspect of the present disclosure, there is provided an information processing method executed by a computer, the method comprising: acquiring first information including biological information of a user; determining whether or not the biological information included in the first information satisfies a predetermined condition, and issuing, to a measurement apparatus that measures biological information of the user and that is not associated with the computer, a command regarding the measurement according to a result of the determination.

According to a twentieth aspect of the present disclosure, there is provided an information processing program for causing a computer to execute processing comprising: acquiring first information including biological information of a user; determining whether or not the biological information included in the first information satisfies a predetermined condition; and issuing, to a measurement apparatus that measures biological information of the user and that is not associated with the computer, a command regarding the measurement according to a result of the determination.

According to the above aspects, the information processing apparatus, the information processing system, the information processing method, and the information processing program of the present disclosure can acquire the biological information as necessary according to the health condition of the user.

### BRIEF DESCRIPTION OF THE DRAWINGS

FIG. 1 is a schematic configuration diagram of an information processing system.

FIG. 2 is a block diagram illustrating an example of a hardware configuration of an information processing apparatus.

FIG. 3 is a block diagram illustrating an example of a functional configuration of the information processing apparatus.

FIG. 4 is an example of a table in which types of first biological information and second biological information according to a user's intention are defined.

FIG. 5 is an example of a table in which a command for a second measurement apparatus according to first information is defined.

FIG. 6 is a flowchart illustrating an example of information processing.

FIG. 7 is a schematic configuration diagram illustrating a modified example of the information processing system.

FIG. 8 is a schematic configuration diagram illustrating a modified example of the information processing system.

FIG. 9 is an example of a table in which a command for a third measurement apparatus according to the second biological information is defined.

### DETAILED DESCRIPTION

Hereinafter, exemplary embodiments for implementing the technology of the present disclosure will be described in detail with reference to the drawings.

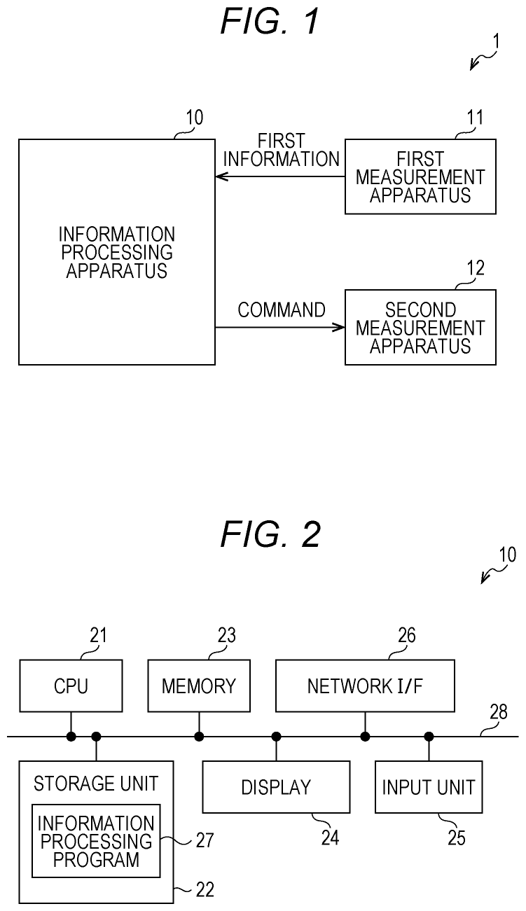

An example of a configuration of an information processing system 1 according to the present exemplary embodiment will be described with reference to FIG. 1. As illustrated in FIG. 1, the information processing system 1 includes an information processing apparatus 10, at least one first measurement apparatus 11, and at least one second measurement apparatus 12. The information processing apparatus 10 and the first measurement apparatus 11 can communicate with each other via wired or wireless communication. The information processing apparatus 10 and the second measurement apparatus 12 can communicate with each other via wired or wireless communication. As a communication method of the wireless communication, a known short-range wireless communication method such as Bluetooth (registered trademark), a Bluetooth low energy (BLE) beacon, and a radio frequency identifier (RFID) can be appropriately applied.

Each of the first measurement apparatus 11 and the second measurement apparatus 12 has a function of measuring biological information of a user. Hereinafter, biological information that is measured by the first measurement apparatus 11 is referred to as “first biological information”, and biological information that is measured by the second measurement apparatus 12 is referred to as “second biological information”. In a case where the first biological information and the second biological information are not distinguished from each other, the first biological information and the second biological information are simply referred to as “biological information”. In a case where the first measurement apparatus 11 and the second measurement apparatus 12 are not distinguished from each other, the first measurement apparatus 11 and the second measurement apparatus 12 are simply referred to as “measurement apparatus”.

The biological information may be, for example, information indicating at least one of a body temperature, a heart rate, an electrocardiogram, an electromyogram, blood pressure, arterial blood oxygen saturation (SpO2), a weight, a body fat percentage, a muscle mass, a bone density, a blood glucose level, or the like. In these cases, as each of the measurement apparatuses, for example, a thermometer, a heart rate meter, a body composition meter, a blood glucose self-measuring device, and a wearable terminal such as a smart watch including a sensor that measures biological information such as a heart rate and arterial blood oxygen saturation can be used.

Further, for example, the biological information may be information indicating results of various examinations such as a hematological examination, a biochemical examination, an infectious disease examination, and a urine examination. The hematological examination is an examination in which, for example, the number of white blood cells, the number of red blood cells, hemoglobin concentration, and the like are obtained as examination results. The biochemical examination is, for example, an examination in which various indicators regarding enzymes, proteins, sugars, lipids, electrolytes, and the like are obtained as examination results. The infectious disease examination is, for example, an examination in which the presence or absence of various infectious diseases such as influenza infection and COVID-19, is obtained as examination results. The urine examination is, for example, an examination in which urine sugar, urine protein, urine occult blood, and the like are obtained as examination results. In these cases, as each of the measurement apparatuses, for example, a known analysis apparatus that performs analysis using blood, urine, or the like as an analyte can be used.

Further, for example, the biological information may be information indicating action histories of the user, such as the number of steps, a sleeping time, an exercise time, and a walking time. In these cases, for example, a wearable terminal such as a smart watch including an acceleration sensor capable of detecting an action of the user, a smartphone, or the like can be used as each of the measurement apparatuses.

As illustrated in FIG. 1, the first measurement apparatus 11 transmits first information including the measured first biological information to the information processing apparatus 10. The information processing apparatus 10 acquires the first information including the first biological information of the user from the first measurement apparatus 11. In addition, the information processing apparatus 10 issues, to the second measurement apparatus 12, a command regarding measurement of the second biological information according to the first biological information.

The second measurement apparatus 12 measures the second biological information in response to the command from the information processing apparatus 10. The second measurement apparatus 12 is a measurement apparatus not associated with the information processing apparatus 10. The term “not associated” means that connection is established between the information processing apparatus 10 and the second measurement apparatus 12 only at the time of communication for issuing a command from the information processing apparatus 10 to the second measurement apparatus 12, and that the connection is not established and presetting for the connection is not performed in other periods. For example, in a case where the communication method is Bluetooth, the information processing apparatus and the second measurement apparatus 12 perform pairing only at the time of communication, and cancel the pairing after the communication. Accordingly, in a case where the information processing apparatus 10 and the second measurement apparatus 12 communicate with each other again, the pairing needs to be performed again.

Although FIG. 1 illustrates one first measurement apparatus 11 and one second measurement apparatus 12, the present disclosure is not limited thereto, and the information processing system 1 may include a plurality of first measurement apparatuses 11 and a plurality of second measurement apparatuses 12. In addition, the plurality of first measurement apparatuses 11 may measure the same type of first biological information or may measure different types of first biological information. Similarly, the plurality of second measurement apparatuses 12 may measure the same type of second biological information or may measure different types of second biological information.

Hereinafter, a detailed configuration of the information processing apparatus 10 will be described. First, an example of a hardware configuration of the information processing apparatus 10 according to the present exemplary embodiment will be described with reference to FIG. 2. As illustrated in FIG. 2, the information processing apparatus 10 includes a central processing unit (CPU) 21, a nonvolatile storage unit 22, and a memory 23 as a temporary storage area. The information processing apparatus 10 includes a display 24 such as a liquid crystal display, an input unit 25 such as a keyboard, a mouse, and buttons, and a network interface (I/F) 26 that performs wired or wireless communication with the first measurement apparatus 11, the second measurement apparatus 12, and an external network (not illustrated). The CPU 21, the storage unit 22, the memory 23, the display 24, the input unit 25, and the network OF 26 are connected with each other via a bus 28, such as a system bus and a control bus, such that the CPU 21, the storage unit 22, the memory 23, the display 24, the input unit and the network OF 26 can transmit and receive various kinds of information to and from each other. As the information processing apparatus 10, for example, a smartphone, a wearable terminal, a tablet terminal, a personal computer, a server computer, and the like can be used.

The storage unit 22 is implemented by, for example, a storage medium such as a hard disk drive (HDD), a solid state drive (SSD), or a flash memory. An information processing program 27 is stored in the storage unit 22 in the information processing apparatus 10. The CPU 21 reads the information processing program 27 from the storage unit 22, loads the information processing program 27 into the memory 23, and executes the loaded information processing program 27. The CPU 21 is an example of a processor according to the present disclosure.

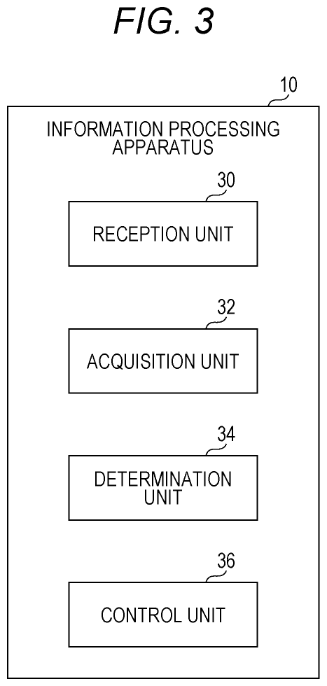

Next, an example of a functional configuration of the information processing apparatus according to the exemplary embodiment will be described with reference to FIG. 3. As illustrated in FIG. 3, the information processing apparatus 10 includes a reception unit 30, an acquisition unit 32, a determination unit 34, and a control unit 36. The CPU 21 functions as the reception unit 30, the acquisition unit 32, the determination unit 34, and the control unit 36 by executing the information processing program 27.

The reception unit 30 receives designation by the user for at least one of a type of the first biological information acquired from the first measurement apparatus 11 (that is, to be used for determination by the determination unit 34 to be described later) or a type of the second biological information as a candidate for measurement by the second measurement apparatus 12. Specifically, the reception unit 30 may receive a user's intention regarding health management of the user and set at least one of the type of the first biological information or the type of the second biological information according to the received intention.

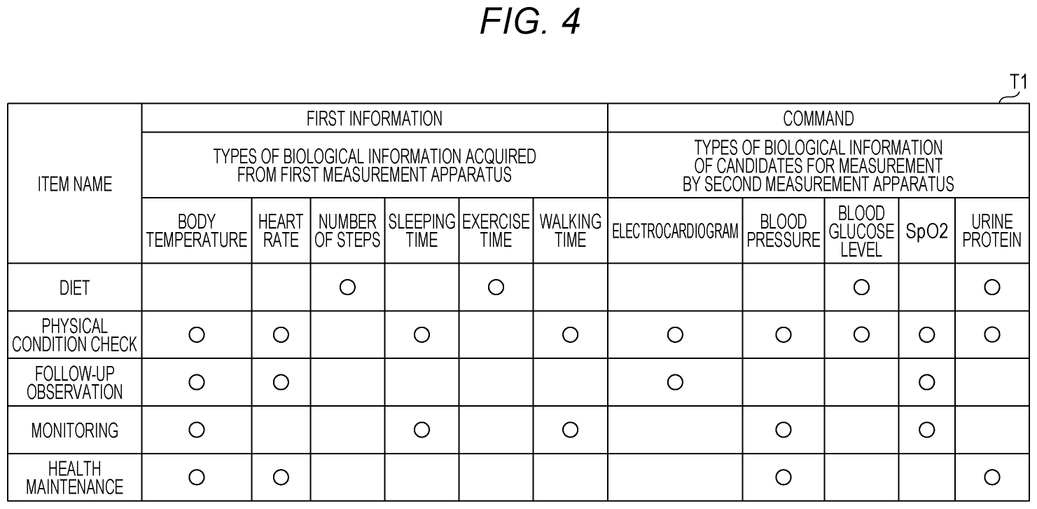

FIG. 4 illustrates an example of a table T1 in which user's intentions regarding health management of the user, and the first biological information to be acquired and the second biological information as candidates for measurement, which are set for each intention, are predetermined. The table T1 is a predetermined table, and is stored in, for example, the storage unit 22. In FIG. 4, “diet” is an item for the purpose of confirming the effects of exercise and dietary improvement. “Physical condition check” is an item for the purpose of checking whether or not the physical condition has deteriorated due to overwork, lack of sleep, lack of exercise, or the like. “Follow-up observation” is an item for the purpose of follow-up observation in the prognosis of a disease. “Monitoring” is an item for the purpose of checking the health conditions of children and the elderly. “Health maintenance” is an item for the purpose of early detection in a case where persons are affected with diseases such as heat stroke and infectious diseases by confirming whether or not the healthy states can be maintained.

The reception unit 30 displays the table T1 on the display 24, and receives the user's intention (selection of “item name” in FIG. 4). The user checks the table T1 displayed on the display 24 and selects an item name according to the user's intention via the input unit 25. The reception unit 30 sets first biological information with a circle in the field of the selected item name as a target to be acquired from the first measurement apparatus 11. Similarly, the reception unit 30 sets second biological information with a circle in the field of the selected item name as a candidate for measurement by the second measurement apparatus 12.

Note that the second biological information set by the reception unit 30 as a candidate for measurement at this point in time has a possibility of being measured, and may not be measured depending on the determination by the determination unit 34 described later. In addition, various kinds of biological information illustrated in FIG. 4 may be acquired from a plurality of different first measurement apparatuses 11 or may be measured by a plurality of different second measurement apparatuses 12.

Hereinafter, it is assumed that the user has selected “health maintenance” in FIG. 4. The reception unit 30 sets a “body temperature” and a “heart rate” as the first biological information to be acquired from the first measurement apparatus 11. The reception unit 30 sets “blood pressure” and “urine protein” as second biological information of candidates for measurement by the second measurement apparatus 12.

The acquisition unit 32 acquires, from the first measurement apparatus 11, the first information including the first biological information of the user according to the types of the first biological information set by the reception unit 30 and to be acquired. As described above, since the first biological information set by the reception unit 30 and to be acquired from the first measurement apparatus 11 is the “body temperature” and the “heart rate”, the acquisition unit 32 acquires the “body temperature” and the “heart rate” as the first information from the first measurement apparatus 11. That is, it suffices for the first information to include at least one type of the first biological information, and the first information may be information including a plurality of pieces of first biological information of different types.

The determination unit 34 determines whether or not the first biological information included in the first information acquired by the acquisition unit 32 satisfies a predetermined condition. Specifically, the determination unit 34 determines whether or not at least one of the plurality of pieces of first biological information (“body temperature” and “heart rate”) included in the first information satisfies the condition.

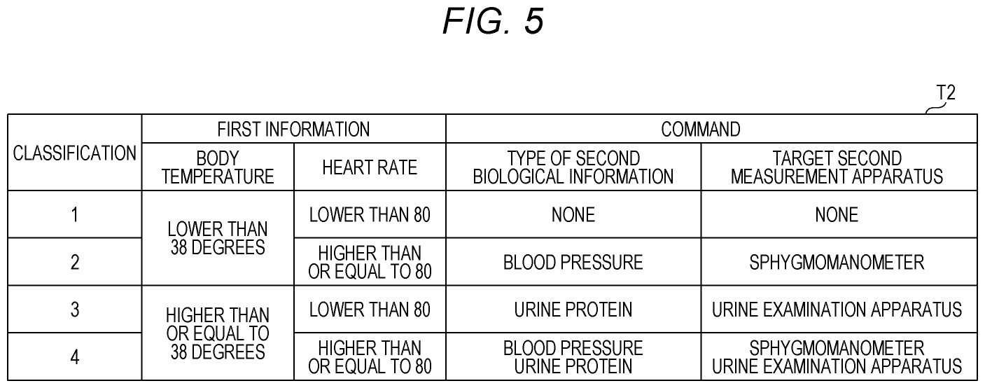

FIG. 5 illustrates an example of a table T2 used for the determination by the determination unit 34. The table T2 is a table corresponding to “health maintenance” of the table T1. In the table T2, classifications are defined in advance according to respective threshold values of the “body temperature” and “heart rate” included in the first information. A table used for the determination by the determination unit 34 is determined in advance for each item name indicated in the table T1, and is stored in advance in, for example, the storage unit 22.

The determination unit 34 collates the “body temperature” and “heart rate” included in the first information with the table T2, and determines to which classification the “body temperature” and “heart rate” included in the first information corresponds. For example, it is assumed that the body temperature acquired by the acquisition unit 32 is equal to or higher than 38 degrees and the heart rate acquired by the acquisition unit 32 is less than 80. In this case, the determination unit 34 collates the body temperature and the heart rate with the table T2 and determines that the classification regarding the first biological information is “classification 3”.

That is, the condition used for the determination by the determination unit 34 may be related to a predetermined threshold value for the first biological information included in the first information. In addition, the condition used for the determination by the determination unit 34 may be determined in advance for each type of the first biological information included in the first information such that threshold values are different for each of the “body temperature” and the “heart rate” in the table T2.

The control unit 36 issues, to the second measurement apparatus 12, a command regarding the measurement of the second biological information according to the result of the determination by the determination unit 34. Specifically, the control unit 36 issues the command according to the classification determined by the determination unit 34. For example, the control unit 36 refers to the table T2 and issues a command instructing the “urine examination apparatus” (second measurement apparatus 12) to measure “urine protein” (second biological information) as a command corresponding to “classification 3” determined by the determination unit 34. That is, the command issued by the control unit 36 to the second measurement apparatus 12 is an instruction to cause the second measurement apparatus 12 to measure the second biological information of the user.

The control unit 36 establishes connection with the second measurement apparatus 12 before issuing the command to the second measurement apparatus 12, and releases the connection with the second measurement apparatus 12 after issuing the command to the second measurement apparatus 12. To “establish the connection” means that the information processing apparatus 10 and the second measurement apparatus 12 are connected with each other in a one to-one manner. For example, when the control unit 36 determines to issue a command instructing the urine examination apparatus to measure urine protein, the control unit 36 performs pairing with the urine examination apparatus by Bluetooth to establish the connection. Thereafter, the control unit 36 issues the command to the urine examination apparatus via Bluetooth, and releases the pairing via Bluetooth after issuing the command to the urine examination apparatus, thereby releasing the connection with the urine examination device.

In a case where a plurality of second measurement apparatuses 12 that measure the second biological information are present (for example, in the case of “classification 4” in the table T2 in FIG. 5), the control unit 36 may issue the command to each of the plurality of second measurement apparatuses 12. Further, the control unit 36 may establish the connection with each of the second measurement apparatuses 12 before issuing the command to each of the plurality of second measurement apparatuses 12, and may release the connection with each of the second measurement apparatuses 12 after issuing the command to each of the second measurement apparatuses 12.

In addition, in a case where a plurality of second measurement apparatuses 12 that measure the same type of second biological information are present (for example, in a case where a plurality of urine examination apparatuses are present), the control unit 36 may search for a second measurement apparatus 12 present near the current location of the user among the second measurement apparatuses 12 and issue the command to the second measurement apparatus 12. In this case, the control unit 36 may guide the user to the searched second measurement apparatus 12 and guide the user to perform the measurement of the second biological information using the second measurement apparatus 12. The search for the second measurement apparatus 12 present near the current location by the control unit 36 can be appropriately carried out, with known methods such as a method using a BLE beacon and a method using a global positioning system (GPS) function.

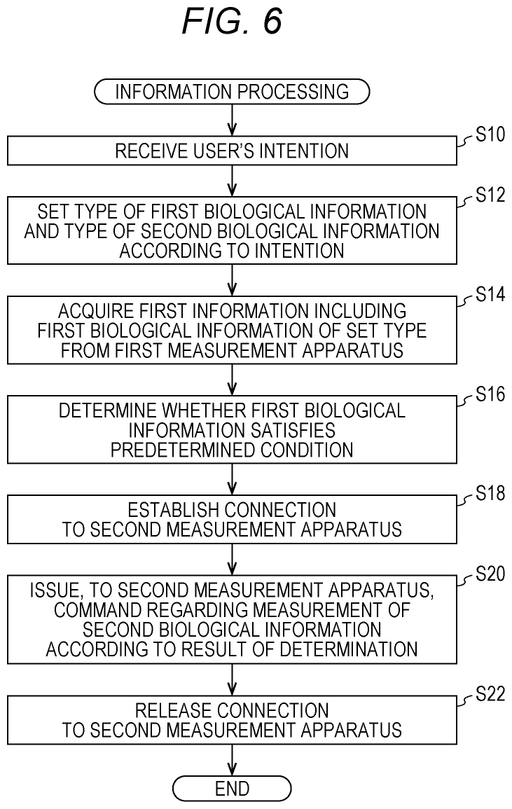

Next, an operation of the information processing apparatus 10 according to the present exemplary embodiment will be described with reference to FIG. 6. In the information processing apparatus 10, the CPU 21 executes the information processing program 27 to execute information processing illustrated in FIG. 6. The information processing is executed, for example, in a case where an instruction to start the execution is input by the user via the input unit 25.

In step S10, the reception unit 30 receives a user's intention regarding health management of the user. In step S12, the reception unit 30 sets a type of the first biological information to be acquired from the first measurement apparatus 11 and a type of the second biological information as a candidate for measurement by the second measurement apparatus 12 according to the intention received in step S10. In step S14, the acquisition unit 32 acquires first information including the first biological information of the type set in step S12 from the first measurement apparatus 11.

In step S16, the determination unit 34 determines whether or not the first biological information included in the first information acquired in step S14 satisfies the predetermined condition. In step S18, the control unit 36 establishes connection with the second measurement apparatus 12 to which a command regarding the measurement of the second biological information has been determined to be issued according to the result of the determination in step S16. In step S20, the control unit 36 issues, to the second measurement apparatus 12 which has established the connection in step S18, the command regarding the measurement of the second biological information according to the result of the determination in step S16. In step S22, the control unit 36 releases the connection with the second measurement apparatus 12, and ends the information processing.

As described above, the information processing apparatus 10 includes at least one processor, and the processor acquires the first information including the first biological information of the user and determines whether or not the first biological information included in the first information satisfies the predetermined condition. In addition, the information processing apparatus 10 issues, according to the result of the determination, a command regarding the measurement of the second biological information to the second measurement apparatus that measures the second biological information of the user and that is not associated with the information processing apparatus 10.

That is, according to the information processing apparatus 10 according to the exemplary embodiment, the second biological information can be acquired as necessary according to the first biological information indicating the health condition of the user. Therefore, for example, in a case where the necessity is high, the measurement of the second biological information is positively performed, and thus it is possible to find a sign of deterioration of a disease condition at an early stage and to use the second biological information for health management. On the other hand, in a case where the necessity is low, the measurement of the second biological information is not positively performed, and thus it is possible to reduce the inconvenience for the user and to suppress the power consumption of the second measurement apparatus 12.

Although the above exemplary embodiment describes the form in which the reception unit 30 receives a user's intention (refer to FIG. 4) regarding health management of the user, and sets a type of the first biological information and a type of the second biological information according to the received intention, the present disclosure is not limited thereto. For example, the reception unit 30 may display a list of types of the first biological information that can be acquired from the first measurement apparatus 11 and types of the second biological information that can be measured by the second measurement apparatus 12 on the display 24 so that the user can arbitrarily select a type.

Further, for example, the information processing apparatus 10 may not have the function of the reception unit 30, and the types of the first biological information that can be acquired from the first measurement apparatus 11 and the types of the second biological information that can be measured by the second measurement apparatus 12 may be determined in advance. That is, the processing of step S10 and step S12 in FIG. 6 may be omitted.

Although the exemplary embodiment describes the form in which one threshold value for the first biological information is defined in advance (see the table T2 illustrated in FIG. 5), the present disclosure is not limited thereto. A plurality of threshold values regarding the first biological information may be defined in a stepwise manner. In this case, the determination unit 34 may make a determination by subdividing the section of the first biological information using the plurality of threshold values defined in a stepwise manner.

Although the above exemplary embodiment describes the form in which the command issued by the control unit 36 to the second measurement apparatus 12 is the instruction to measure the second biological information, the present disclosure is not limited thereto. For example, the control unit 36 may instruct the second measurement apparatus 12 to transmit the second biological information of the user measured by the second measurement apparatus 12 to the information processing apparatus 10 as the command issued to the second measurement apparatus 12.

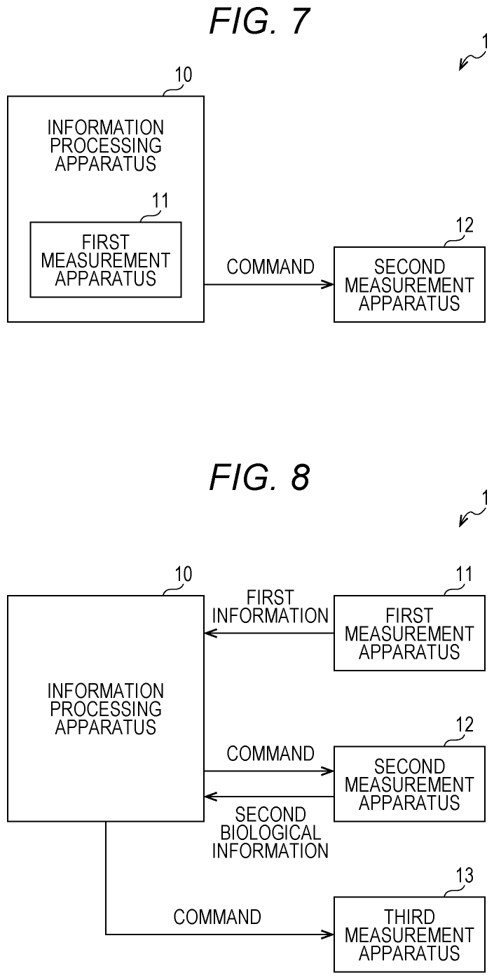

The configuration of the information processing system 1 according to the exemplary embodiment is not limited to the example illustrated in FIG. 1. For example, some or all of the information processing apparatus 10, the first measurement apparatus 11, and the second measurement apparatus 12 included in the information processing system 1 may be the same apparatus. For example, as illustrated in FIG. 7, the information processing system 1 may include an information processing apparatus 10 including the first measurement apparatus 11 that measures the first biological information and at least one second measurement apparatus 12 that measures the second biological information in response to a command from the information processing apparatus 10. For example, as this information processing apparatus a wearable terminal such as a smart watch including a sensor that measures the first biological information such as a heart rate and SpO2 may be used.

Further, for example, the information processing system 1 may include three or more measurement apparatuses. For example, as illustrated in FIG. 8, the information processing system 1 may include the information processing apparatus 10, the first measurement apparatus 11, the second measurement apparatus 12, and a third measurement apparatus 13.

In this case, the information processing apparatus 10 may acquire biological information from each of the measurement apparatuses to issue a command in a chained manner. Specifically, the information processing apparatus 10 issues, according to the first information acquired from the first measurement apparatus 11, a command instructing the second measurement apparatus 12 to measure the second biological information and to transmit the measured second biological information to the information processing apparatus Thereafter, the acquisition unit 32 of the information processing apparatus 10 acquires the second biological information measured by the second measurement apparatus 12 in response to the command, and the determination unit 34 determines whether or not the second biological information satisfies a predetermined condition, and issues a command to the third measurement apparatus 13 different from the second measurement apparatus 12 according to a result of the determination.

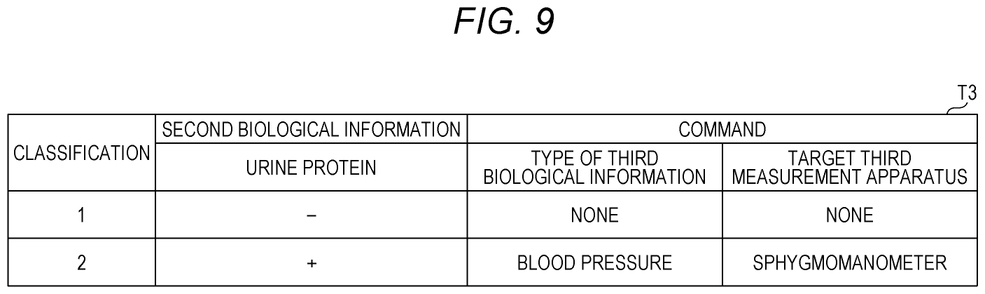

A specific example of such processing will be described with reference to FIG. 9. As exemplified in the above-described exemplary embodiment, it is assumed that the determination unit 34 determines “classification 3” (refer to FIG. 5) according to the body temperature and the heart rate acquired by the acquisition unit 32, and that the control unit 36 issues a command instructing the urine examination apparatus (second measurement apparatus 12) to perform measurement of urine protein (second biological information). In addition, it is assumed that the control unit 36 issues a command to transmit information indicating whether the measured urine protein is negative or positive to the information processing apparatus 10 together with an instruction to measure urine protein. Thereafter, the acquisition unit 32 acquires information indicating whether the urine protein is negative or positive from the second measurement apparatus 12.

FIG. 9 illustrates an example of a table T3 used for the determination by the determination unit 34. In the table T3, classifications are defined in advance according to whether “urine protein” is negative or positive. The determination unit 34 collates the information (second biological information) indicating whether the urine protein acquired by the acquisition unit 32 is negative or positive with the table T3, and determines to which classification the information corresponds. For example, it is assumed that the acquisition unit 32 acquires information indicating that the urine protein is positive. In this case, the determination unit 34 collates the information with the table T3, and determines that the classification regarding the second biological information is “classification 2”. In this case, the control unit 36 refers to the table T3 and issues, as a command corresponding to the “classification 2” determined by the determination unit 34, a command instructing the “sphygmomanometer” (third measurement apparatus 13) to measure “blood pressure” (third biological information).

As described above, since a plurality of pieces of biological information can be acquired in a chained manner in a case where it is particularly necessary according to the health condition of the user, it is possible to early detect a sign of deterioration of a disease condition and to use the biological information for health management.

In addition, in the exemplary embodiment, the information processing apparatus 10 may include, for example, known payment means using cash, a credit card, an integrated circuit (IC) card, a QR code (registered trademark), or the like, and may include a function of causing the user to settle a usage fee. In this case, the information processing apparatus 10 may settle the usage fee before issuing the command to the second measurement apparatus 12.

In the exemplary embodiment, for example, the following various processors can be used as a hardware structure of the processing units that perform the various processes, such as the reception unit 30, the acquisition unit 32, the determination unit 34, and the control unit 36. As described above, in addition to the CPU which is a general-purpose processor that executes software (program) to function as the various processing units, examples of the various processors include a programmable logic device (PLD) which is a processor having a circuit configuration which is changeable after a field programmable gate array (FPGA) or the like is manufactured, and a dedicated electric circuit which is a processor having a circuit configuration designed as a dedicated circuit in order to perform specific processing, such as an application specific integrated circuit (ASIC).

Each of the processing units may be configured by one of these various processors or may be configured by a combination of two or more processors of the same type or different types (for example, a combination of a plurality of FPGAs or a combination of a CPU and an FPGA). In addition, a plurality of processing units may be configured by one processor.

As an example in which a plurality of processing units are configured by one processor, first, as represented by a computer such as a client and a server, there is a form in which one processor is configured by a combination of one or more CPUs and software and this processor functions as the plurality of processing units. Second, as represented by a system on chip (SoC) or the like, there is a form in which a processor that implements the functions of the entire system including the plurality of processing units with one integrated circuit (IC) chip is used. As described above, the various processing units are configured using one or more of the various processors as the hardware structure.

Further, more specifically, an electric circuit (circuitry) in which circuit elements such as semiconductor elements are combined can be used as the hardware structure of the various processors.

Further, although the above-described exemplary embodiment describes the configuration in which the information processing program 27 is stored (installed) in the storage unit 22 in advance, the present disclosure is not limited thereto. The information processing program 27 may be provided in a recorded form on a recording medium such as a compact disc read only memory (CD-ROM), a digital versatile disc read only memory (DVD-ROM), or a Universal Serial Bus (USB) memory. In addition, the information processing program 27 may be downloaded from an external device via a network. Furthermore, the technology of the present disclosure extends to a storage medium that stores the information processing program in a non-transitory manner, in addition to the information processing program.

In the technique of the present disclosure, the above-described exemplary embodiments can be appropriately combined. The details of the above descriptions and illustrations are detailed descriptions of the portions according to the technology of the present disclosure and are merely examples of the technology of the present disclosure. For example, the above description of the configurations, the functions, the operations, and the effects is an example of the configurations, functions, operations, and effects of the portions according to the technology of the present disclosure. Accordingly, it goes without saying that unnecessary portions may be removed, new elements may be added, or replacement may be made with respect to the details of the above descriptions and illustrations without departing from the scope of the technology of the present disclosure.

JP2021-035801 filed on Mar. 5, 2021 is incorporated herein by reference in its entirety. All the documents, patent applications, and technical standards described in this specification are herein incorporated by reference to the same extent as if each individual publication, patent application, or technical standard was specifically and individually indicated to be incorporated by reference.

What is claimed is:

1. An information processing apparatus comprising at least one processor, wherein the processor is configured to:
acquire first information including biological information of a user;
determine whether or not the biological information included in the first information satisfies a predetermined condition; and
issue, to a measurement apparatus that measures the biological information of the user and that is not associated with the information processing apparatus, a command regarding the measurement according to a result of the determination.

2. The information processing apparatus according to claim 1, wherein:
the first information includes a plurality of pieces of different types of biological information,
the condition is predetermined for each type of the biological information, and
the processor is configured to determine whether or not at least one of the plurality of pieces of biological information included in the first information satisfies the condition.

3. The information processing apparatus according to claim 1, wherein the processor is configured to issue, to each of a plurality of measurement apparatuses that measure the same type of biological information, the command according to the result of the determination.

4. The information processing apparatus according to claim 1, wherein the processor is configured to issue, to each of a plurality of measurement apparatuses that measure different types of biological information, the command according to the result of the determination.

5. The information processing apparatus according to claim 1, wherein the command is an instruction to cause the measurement apparatus to measure the biological information of the user.

6. The information processing apparatus according to claim 1, wherein the command is an instruction to transmit the biological information of the user measured by the measurement apparatus to the information processing apparatus.

7. The information processing apparatus according to claim 6, wherein the processor is configured to:
acquire the biological information of the user measured by the measurement apparatus in response to the command;
determine whether or not the biological information satisfies the predetermined condition; and
issue, to a measurement apparatus different from the measurement apparatus, a command regarding the measurement of the biological information of the user according to the result of the determination.

8. The information processing apparatus according to claim 1, wherein the processor is configured to receive designation by the user for at least one of a type of the biological information used for the determination or a type of biological information of a candidate for measurement by the measurement apparatus.

9. The information processing apparatus according to claim 1, wherein the processor is configured to:
receive a user's intention regarding health management of the user; and
set at least one of a type of the biological information used for the determination or a type of biological information of a candidate for measurement by the measurement apparatus according to the user's intention.

10. The information processing apparatus according to claim 1, wherein the condition relates to a predetermined threshold value for the biological information included in the first information.

11. The information processing apparatus according to claim 10, wherein:
a plurality of threshold values are defined in a stepwise manner, and
the processor is configured to:
determine a classification of the biological information included in the first information using the plurality of threshold values; and
issue the command according to the classification.

12. The information processing apparatus according to claim 1, wherein the processor is configured to:
establish connection with the measurement apparatus before issuing the command to the measurement apparatus; and
release the connection with the measurement apparatus after issuing the command to the measurement apparatus.

13. The information processing apparatus according to claim 1, wherein the processor is configured to:
search for the measurement apparatus present near a current location of the user; and
issue the command to the measurement apparatus.

14. The information processing apparatus according to claim 1, wherein the processor is configured to settle a usage fee before issuing the command to the measurement apparatus.

15. The information processing apparatus according to claim 1, wherein the biological information indicates at least one of a body temperature, a heart rate, an electrocardiogram, an electromyogram, blood pressure, arterial blood oxygen saturation, a weight, a body fat percentage, a muscle mass, a bone density, a blood glucose level, a result of a hematological examination, a result of an infectious disease examination, a result of a biochemical examination, or a result of a urine examination.

16. The information processing apparatus according to claim 1, further comprising a first measurement apparatus that measures biological information of the user,
wherein the processor is configured to acquire the first information including the biological information of the user measured by the first measurement apparatus.

17. An information processing system comprising:
the information processing apparatus according to claim 16; and
at least one second measurement apparatus that measures biological information of the user in response to a command from the information processing apparatus.

18. An information processing system comprising:
the information processing apparatus according to claim 1;
at least one first measurement apparatus that measures biological information of the user and that transmits the first information including the measured biological information to the information processing apparatus; and
at least one second measurement apparatus that measures biological information of the user in response to a command from the information processing apparatus.

19. An information processing method executed by a computer, the method comprising:
acquiring first information including biological information of a user;
determining whether or not the biological information included in the first information satisfies a predetermined condition; and
issuing, to a measurement apparatus that measures biological information of the user and that is not associated with the computer, a command regarding the measurement according to a result of the determination.

20. A non-transitory computer-readable storage medium storing an information processing program for causing a computer to execute processing comprising:
acquiring first information including biological information of a user;
determining whether or not the biological information included in the first information satisfies a predetermined condition; and
issuing, to a measurement apparatus that measures biological information of the user and that is not associated with the computer, a command regarding the measurement according to a result of the determination.
